(19) United States
(12) Patent Application Publication
(10) Pub. No.: US 2026/0263034 A1
(43) Pub. Date: Sep. 10, 2026

(54) METHOD AND SYSTEM FOR SCREENING SPACEFLIGHT-ASSOCIATED NEURO-OCULAR SYNDROME AND APPLICATIONS OF SAME

(71) Applicant: AUGUSTA UNIVERSITY RESEARCH INSTITUTE, INC., Augusta, GA (US)

(72) Inventor: Matthew Lyon, North Augusta, SC (US)

(21) Appl. No.: 18/993,628

(22) PCT Filed: Jul. 12, 2023
(86) PCT No.: PCT/US2023/027485

§ 371 Date: Jan. 13, 2025

Related U.S. Application Data
(60) Provisional application No. 63/389,078, filed on Jul. 14, 2022.

(51) Int. Cl. A61B 8/10; A61B 8/00; A61B 8/08; G06T 7/00; G06T 7/62

(52) U.S. Cl. CPC A61B 8/10; A61B 8/08; A61B 8/4427; A61B 8/4477; A61B 8/483; A61B 8/485; G06T 7/0014; G06T 7/62; G06T 2200/04; G06T 2207/10136; G06T 2207/30041

(57) ABSTRACT

One aspect of the invention relates to a method for screening SANS for a living subject comprising obtaining 3D images or 3D quantitative measurements of at least one optic nerve sheath of the living subject at rest and with a provocative maneuver, respectively, prior to a spaceflight, wherein the 3D images or the 3D quantitative measurements obtained at rest is used as a baseline; evaluating the 3D images or the 3D quantitative measurements obtained at rest and with the provocative maneuver; and determining if the living subject is at risk of developing the SANS based on the evaluation of the 3D images or the 3D quantitative measurements.

### CROSS-REFERENCE TO RELATED PATENT APPLICATION

This application claims priority to and the benefit of U.S. Provisional Application Ser. No. 63/389,078, filed Jul. 14, 2022, which is incorporated herein by reference in its entirety.

### FIELD OF THE INVENTION

This invention relates generally to medical diagnosis, and more particularly to a method and a system for screening spaceflight-associated neuro-ocular syndrome (SANS) and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

On Earth, gravity causes the fluid in the body to shift towards the ground, aiding in the drainage of fluid from the head. Our human bodies are adapted to gravity as a mechanism to aid in venous drainage, and there are no active physiological mechanisms to actively remove fluid from the head. It has been discovered that the structural components of the optic nerve sheath (ONS) can be damaged by various mechanisms. Once these structures are damaged, they cannot hold the optic nerve (ON) tightly to the sheath, resulting in the loss of the sheath's ability to resist expansion from an increased volume of cerebrospinal fluid (CSF). In a gravity environment, such as on Earth, fluid naturally drains from the ONS due to gravity. Hence, there are no known deleterious effects or pathologies resulting from this loss of ONS structural integrity. In space, fluid does not have gravity's assistance in draining fluid from the head. Therefore, in patients with damaged ONS structures, the fluid can accumulate in the ONS.

Spaceflight-Associated Neuro-ocular Syndrome (SANS) is a constellation of findings and symptoms that have been found in astronauts who have undergone long duration space flight (LDSF) mission in microgravity environments. The SANS Evidence Report presents detailed documentation of the “potential risk of permanent ocular changes largely as a result of globe flattening during space flight”. The report states that, “It is thought that the ocular structural and optic nerve changes are caused by events precipitated by the cephalad fluid shift crew members experience during long-duration spaceflight. It is believed some crew members are more susceptible to these changes because of their genetic/anatomical predisposition or lifestyle (fitness) related factors”. It is reported that 37 to 51% of long-duration crew members demonstrate one or more SANS symptoms. The SANS report concludes without a proven explanation or cause for the development of SANS.

Although researchers have been able to document the presence of SANS, a major limitation of prior SANS research has been the ability to measure dynamic changes in the ONS and the effects on retinal and optic nerve perfusion in real-time. Studies have tried to replicate and study SANS using bed rest, the most prevalent analog of space flight, yet this approach has numerous well-documented limitations.

Therefore, a heretofore unaddressed need exists in the art to address the aforementioned deficiencies and inadequacies.

### SUMMARY OF THE INVENTION

In view of the foregoing, one of the objectives of this invention is to provide a unique mechanism of spaceflight-associated neuro-ocular syndrome (SANS), a mechanism of measuring the predisposing to developing SANS prior to space flight and measuring the development of SANS, a mechanism of monitoring for the efficacy of countermeasures, and a mechanism for detecting ongoing injury during space flight.

In one aspect, the invention relates to a method for screening spaceflight-associated neuro-ocular syndrome (SANS) for a living subject. The method comprises obtaining three-dimensional (3D) images or 3D quantitative measurements of at least one optic nerve sheath (ONS) of the living subject at rest and with a provocative maneuver, respectively, prior to a spaceflight, wherein the 3D images or the 3D quantitative measurements obtained at rest is used as a baseline; evaluating the 3D images or the 3D quantitative measurements obtained at rest and with the provocative maneuver; and determining if the living subject is at risk of developing the SANS based on the evaluation of the 3D images or the 3D quantitative measurements.

In one embodiment, the provocative maneuver is an activity that increases the volume in the ONS, causing the ONS to dilate with minimal increases in the intracranial pressure (ICP).

In one embodiment, the provocative maneuver is a Valsalva maneuver.

In one embodiment, the 3D images are 3D ultrasound images obtained by an ultrasound probe, and wherein the 3D quantitative measurements are obtained by a magnetic resonance imaging (MRI) or computed tomography (CT) scanner.

In one embodiment, said evaluating the 3D images or the 3D derived quantitative measurements comprises evaluating a structural integrity of the ONS using the 3D images or the 3D quantitative measurements obtained with the provocative maneuver, by comparing them with the baseline, wherein the structural integrity is a diagnostic and prognostic indicator utilized in the evaluation of the SANS.

In one embodiment, said evaluating the structural integrity of the ONS comprises obtaining changes in a surface area of the ONS by comparing the 3D images or the 3D derived quantitative measurements obtained with the provocative maneuver with the baseline.

In one embodiment, said evaluating the structural integrity of the ONS comprises obtaining changes in a volume of the ONS by comparing the 3D images or the 3D derived quantitative measurements obtained with the provocative maneuver with the baseline.

In one embodiment, said evaluating the structural integrity of the ONS comprises obtaining changes in a surface structure of the ONS by comparing the 3D images or the 3D derived quantitative measurements obtained with the provocative maneuver with the baseline.

In one embodiment, said obtaining the changes in the surface structure of the ONS comprises obtaining a degree of irregularity of the surface structure of the ONS visualized in the 3D images or the 3D derived quantitative measurements obtained with the provocative maneuver.

In one embodiment, the irregularity of the surface structure of the ONS includes bulges, wrinkles, and/or ripples formed on the exterior surface of the ONS.

In one embodiment, the degree of irregularity of the surface structure of the ONS is associated with a severity of a structural damage of the ONS, wherein the severity of the structural damage is proportional to the degree of the irregularity of the surface structure, and the more variability of the surface structure of the ONS, the more underlying structural damage and the higher the risk of developing the SANS in a low-or no-gravity environment.

In one embodiment, the variability of the surface structure of the ONS is measured by one or more of a mean radius at a specified point in the ONS distal to the retina and a standard deviation in the radii; a volume at a specific level measured posterior to the retina; and a volume of the ONS compared to an ideal cylinder.

In one embodiment, the 3D images or the 3D quantitative measurements are of real-time elastography of the ONS, wherein said evaluating the 3D images or the 3D quantitative measurements comprises obtaining changes in a pressure of fluid in the ONS using the 3D images or the 3D quantitative measurements.

In one embodiment, said determining if the living subject is at risk of developing the SANS comprises determining a degree of predisposition to developing the SANS based on the structural integrity of the ONS.

In one embodiment, the degree of predisposition to developing the SANS includes a relative propensity of developing the SANS based on exposure to a low-or zero-gravity environment.

In one embodiment, said determining the degree of predisposition to developing the SANS comprises correlating a dose of low gravity and/or a duration of exposing to a low-or zero-gravity environment to the risk of developing the SANS.

In one embodiment, the method further comprises obtaining 3D images or 3D quantitative measurements of the ONS of the living subject during the spaceflight and/or on a space station in a low-or no-gravity environment; evaluating the 3D image or the 3D quantitative measurement obtained during the spaceflight and/or on the space station in the low-or no-gravity environment by comparing them with the baseline; and determining a development of the SANS, an efficacy of SANS treatments or countermeasures, and/or an ongoing injury during the spaceflight and/or on the space station in the low-or no-gravity environment based on the evaluation of the 3D images or the 3D quantitative measurements.

In one embodiment, said evaluating the 3D images or the 3D derived quantitative measurements comprises evaluating a structural integrity of the ONS using the 3D images or the 3D quantitative measurements obtained during the spaceflight and/or on the space station in the low-or no-gravity environment, by comparing them with the baseline, wherein the structural integrity is a diagnostic and prognostic indicator utilized in the evaluation of the SANS.

In one embodiment, said evaluating the structural integrity of the ONS comprises obtaining changes in one or more of a surface area, a volume, a surface structure, and a pressure of the ONS by comparing the 3D images or the 3D derived quantitative measurements obtained with the provocative maneuver with the baseline.

In one embodiment, said determining if the living subject is at risk of developing the SANS comprises determining the development of the SANS, the efficacy of SANS treatments or countermeasures, and/or the ongoing injury during the spaceflight and/or on the space station in the low-or no-gravity environment based on the structural integrity of the ONS.

In one embodiment, the method further comprises obtaining 3D images or 3D quantitative measurements of the ONS of the living subject after returned to the Earth in a normal gravity environment; evaluating the 3D image or the 3D quantitative measurement obtained after returned on the Earth in the normal gravity environment by comparing them with the baseline; and assessing accumulated damage of the ONS in the low-or no-gravity environment from the baseline, based on the evaluation of the 3D images or the 3D quantitative measurements, and/or resolution of SANS predisposing factors after returned to the Earth in the normal gravity environment.

In another aspect, the invention relates to a method for screening SANS for a living subject comprising obtaining 3D images or 3D quantitative measurements of at least one ONS of the living subject at rest in a normal gravity environment prior to a spaceflight and during the spaceflight and/or on a space station in a low-or no-gravity environment, respectively, wherein the 3D images or the 3D quantitative measurements obtained at rest in the normal gravity environment is used as a baseline; evaluating the 3D image or the 3D quantitative measurement obtained during the spaceflight and/or on the space station in the low-or no-gravity environment by comparing them with the baseline; and determining a degree of predisposition to developing the SANS, an efficacy of SANS treatments or countermeasures, and/or an ongoing injury during the spaceflight and/or on the space station in the low-or no-gravity environment based on the evaluation of the 3D images or the 3D quantitative measurements.

In one embodiment, the 3D images are 3D ultrasound images obtained by an ultrasound probe, and wherein the 3D quantitative measurements are obtained by magnetic an MRI or CT scanner.

In one embodiment, said evaluating the 3D images or the 3D derived quantitative measurements comprises evaluating a structural integrity of the ONS using the 3D images or the 3D quantitative measurements obtained with the provocative maneuver, by comparing them with the baseline, wherein the structural integrity is a diagnostic and prognostic indicator utilized in the evaluation of the SANS.

In one embodiment, said evaluating the structural integrity of the ONS comprises obtaining changes in one or more of a surface area, a volume, a surface structure, and a pressure of the ONS by comparing the 3D images or the 3D derived quantitative measurements obtained with the provocative maneuver with the baseline.

In one embodiment, said obtaining the changes in the surface structure of the ONS comprises obtaining a degree of irregularity of the surface structure of the ONS visualized in the 3D images or the 3D derived quantitative measurements obtained with the provocative maneuver.

In one embodiment, the irregularity of the surface structure of the ONS includes bulges, wrinkles, and/or ripples formed on the exterior surface of the ONS.

In one embodiment, the degree of irregularity of the surface structure of the ONS is associated with a severity of a structural damage of the ONS, wherein the severity of the structural damage is proportional to the degree of the irregularity of the surface structure, and the more variability of the surface structure of the ONS, the more underlying structural damage and the higher the risk of developing the SANS in a low-or no-gravity environment.

In one embodiment, the variability of the surface structure of the ONS is measured by one or more of a mean radius at a specified point in the ONS distal to the retina and a standard deviation in the radii; a volume at a specific level measured posterior to the retina; and a volume of the ONS compared to an ideal cylinder.

In one embodiment, said determining if the living subject is at risk of developing the SANS comprises determining a degree of predisposition to developing the SANS based on the structural integrity of the ONS.

In one embodiment, the degree of predisposition to developing the SANS includes a relative propensity of developing the SANS based on exposure to a low-or zero-gravity environment.

In one embodiment, said determining the degree of predisposition to developing the SANS comprises correlating a dose of low gravity and/or a duration of exposing to a low-or zero-gravity environment to the risk of developing the SANS.

In one embodiment, the method further comprises obtaining 3D images or 3D quantitative measurements of the ONS of the living subject after returned to the Earth in a normal gravity environment; evaluating the 3D image or the 3D quantitative measurement obtained after returned on the Earth in the normal gravity environment by comparing them with the baseline; and assessing accumulated damage of the ONS in the low-or no-gravity environment from the baseline, based on the evaluation of the 3D images or the 3D quantitative measurements, and/or resolution of SANS predisposing factors after returned to the Earth in the normal gravity environment.

In yet another aspect, the invention relates to a system for screening SANS for a living subject. The system comprises a means for obtaining 3D images or 3D quantitative measurements of at least one ONS of the living subject at rest and with a provocative maneuver, respectively, prior to a spaceflight, wherein the 3D images or the 3D quantitative measurements obtained at rest is used as a baseline; and a processor for evaluating the 3D images or the 3D quantitative measurements obtained at rest and with the provocative maneuver, and determining if the living subject is at risk of developing the SANS based on the evaluation of the 3D images or the 3D quantitative measurements.

In one embodiment, the provocative maneuver is an activity that increases the volume in the ONS, causing the ONS to dilate with minimal increases in the ICP.

In one embodiment, the provocative maneuver is a Valsalva maneuver.

In one embodiment, the means comprises an ultrasound probe, an MRI or CT scanner.

In one embodiment, the ultrasound probe is a portable ultrasound using three linear arrays to simultaneously images of the ONS allowing for reconstruction of the 3D image.

In one embodiment, said evaluating the 3D images or the 3D derived quantitative measurements comprises evaluating a structural integrity of the ONS using the 3D images or the 3D quantitative measurements obtained with the provocative maneuver, by comparing them with the baseline, wherein the structural integrity is a diagnostic and prognostic indicator utilized in the evaluation of the SANS.

In one embodiment, said evaluating the structural integrity of the ONS comprises obtaining changes in one or more of a surface area, a volume, a surface structure, and a pressure of the ONS by comparing the 3D images or the 3D derived quantitative measurements obtained with the provocative maneuver with the baseline.

In one embodiment, said obtaining the changes in the surface structure of the ONS comprises obtaining a degree of irregularity of the surface structure of the ONS visualized in the 3D images or the 3D derived quantitative measurements obtained with the provocative maneuver.

In one embodiment, the irregularity of the surface structure of the ONS includes bulges, wrinkles, and/or ripples formed on the exterior surface of the ONS.

In one embodiment, the degree of irregularity of the surface structure of the ONS is associated with a severity of a structural damage of the ONS, wherein the severity of the structural damage is proportional to the degree of the irregularity of the surface structure, and the more variability of the surface structure of the ONS, the more underlying structural damage and the higher the risk of developing the SANS in a low-or no-gravity environment.

In one embodiment, the variability of the surface structure of the ONS is measured by one or more of a mean radius at a specified point in the ONS distal to the retina and a standard deviation in the radii; a volume at a specific level measured posterior to the retina; and a volume of the ONS compared to an ideal cylinder.

In one embodiment, said determining if the living subject is at risk of developing the SANS comprises determining a degree of predisposition to developing the SANS based on the structural integrity of the ONS.

In one embodiment, the degree of predisposition to developing the SANS includes a relative propensity of developing the SANS based on exposure to a low-or zero-gravity environment.

In one embodiment, said determining the degree of predisposition to developing the SANS comprises correlating a dose of low gravity and/or a duration of exposing to a low-or zero-gravity environment to the risk of developing the SANS.

In one embodiment, the means is further adapted for obtaining 3D images or 3D quantitative measurements of the ONS of the living subject during the spaceflight and/or on a space station in a low-or no-gravity environment; evaluating the 3D image or the 3D quantitative measurement obtained during the spaceflight and/or on the space station in the low-or no-gravity environment by comparing them with the baseline; and determining a development of the SANS, an efficacy of SANS treatments or countermeasures, and/or an ongoing injury during the spaceflight and/or on the space station in the low-or no-gravity environment based on the evaluation of the 3D images or the 3D quantitative measurements.

In one embodiment, said evaluating the 3D images or the 3D derived quantitative measurements comprises evaluating a structural integrity of the ONS using the 3D images or the 3D quantitative measurements obtained during the spaceflight and/or on the space station in the low-or no-gravity environment, by comparing them with the baseline, wherein the structural integrity is a diagnostic and prognostic indicator utilized in the evaluation of the SANS.

In one embodiment, said evaluating the structural integrity of the ONS comprises obtaining changes in one or more of a surface area, a volume, a surface structure, and a pressure of the ONS by comparing the 3D images or the 3D derived quantitative measurements obtained with the provocative maneuver with the baseline.

In one embodiment, said determining if the living subject is at risk of developing the SANS comprises determining the development of the SANS, the efficacy of SANS treatments or countermeasures, and/or the ongoing injury during the spaceflight and/or on the space station in the low-or no-gravity environment based on the structural integrity of the ONS.

In one embodiment, the means is further adapted for obtaining 3D images or 3D quantitative measurements of the ONS of the living subject after returned to the Earth in a normal gravity environment; evaluating the 3D image or the 3D quantitative measurement obtained after returned on the Earth in the normal gravity environment by comparing them with the baseline; and assessing accumulated damage of the ONS in the low-or no-gravity environment from the baseline, based on the evaluation of the 3D images or the 3D quantitative measurements, and/or resolution of SANS predisposing factors after returned to the Earth in the normal gravity environment.

These and other aspects of the present invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIGS. 1A-1C show schematically structures of the optic nerve (ON), trabecula (T) and optic nerve sheath (ONS). FIG. 1A: The ONS surrounds the ON and contains cerebrospinal fluid (CSF). FIG. 1B: The ONS is connected to the ON by structural components called trabecula, septa, and pillars. FIG. 1C: A scanning electron microscopy image of the ON, T and ONS.

FIGS. 2A-2B are scanning electron microscopy images of the trabecula of the ONS. FIG. 2A: The normal appearance of the trabecula. FIG. 2B: The trabecula after damage. In this case an increased intracranial pressure was the cause of the damage.

FIGS. 3A-3B are 3D ultrasound images of the ONS in an exemplary experimental porcine model. FIG. 3A: The baseline appearance of the ONS showing a smooth surface. FIG. 3B: A damaged ONS of the same individual. The damage in this case was caused by an elevation in the intracranial pressure.

FIG. 4 shows influence of microgravity on the dynamic ONS structural and functional alterations before and after head injury. A. Normal conditions, a provocative maneuver (PM), such as Valsalva, has no significant effect on the ONS size. Consequently, at the end of the PM, the ONS diameter is comparable to normal. B. In individuals with a history of prior head injury, an elevated ICP event causes structural damage to the ONS. Here, in response to a PM, the ONS cannot resist the change in volume and dilates. Once the PM is stopped, the ONS returns to it's baseline size. In space, the microgravity is the PM, simulating a continuous Valsalva. C. In an individual with a damaged ONS, microgravity/PM will exaggerate the volume in the ONS due to the lax structure. The resulting increase in local pressure will compress venules, increase vascular resistance and capillary hydraulic pressure affecting both blood flow and capillary permeability in the optic nerve and retina. These deleterious events will compromise the optic nerve and retinal function. The sustained ONS diameter distension establishes a positive feedback loop which, with time, exacerbates SANS pathology and symptoms. D. In an individual with no prior head injury, microgravity/PM and the lack of lax ONS structure could either worsen or protect the optic nerve and retina.

FIG. 5 is a 3D ultrasound image of a human with a severely damaged ONS. The severity of the damage is proportional to the degree of the irregularity of the surface structure seen in the image. The more variability of the surface, the more underlying structural damage and the higher the risk of developing SANS in a low gravity environment. This variability can be objectively measured in many ways: 1) mean radius at a specified point in the ONS distal to the retina and the standard deviation in the radii, 2) volume at a specific level measured posterior to the retina (for example the ONS volume 2 mm to 4 mm posterior to the retina, 3) volume of the ONS compared to an ideal cylinder. All these measurements would require a pre-measurement compared to a post Valsalva measurement. The difference pre/post would be proportional to the ONS structural damage with the absolute measures given in any of the methods above. For example: pre-Valsalva the volume is 0.157 ml and the post Valsalva volume is 0.211 ml. This difference in volume of 0.054 ml is consistent with mild ONS damage and the risk of SANS is unlikely with prolonged exposure to a low gravity environment.

FIGS. 6A-6B show a specially designed bedside/portable ultrasound using 3 linear arrays (FIG. 6A) to simultaneously image the ONS (FIG. 6B) allowing for reconstruction of the 3D volume. Using the 3D volume of the ONS, we create 3D images of the ONS and/or a quantitative measurement of the structural damage and volume of the ONS.

FIG. 7 is a real-time elastography of the ONS. The colors are qualitative indicators (quantitative measurements are reported in kPa) of the stiffness and consequentially the amount of pressure on the optic nerve sheath. The qualitative colors are used to indicate there is a pressure on the ONS and there should be increased monitoring for ONS damage. The qualitative measurement is used to give the exact pressure on the ONS with a higher pressure indicating that there is ongoing potential for ONS damage and the rate of the damage. Conversely, the qualitative and quantitative measurement of the ONS can be used to determine the effectiveness of SANS treatment or countermeasures or be used to adjust (tailor) the treatment to the individual.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this specification will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term are the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures. is turned over, elements described as being on the “lower” side of other elements would then be oriented on “upper” sides of the other elements. The exemplary term “lower”, can, therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this specification, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and this specification, and will not be interpreted in an idealized or overly formal sense unless expressly so defined herein.

As used in this specification, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this specification, the phrase “at least one of A, B, and C” should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in a different order (or concurrently) without altering the principles of the invention.

Spaceflight-Associated Neuro-ocular Syndrome (SANS) is a constellation of findings and symptoms that have been found in astronauts who have undergone long duration space flight (LDSF) mission in microgravity environments. Although the specific etiology of the optic disc edema, globe flattening, retinal and choroidal folds and hyperopic shifts described in SANS is unclear, two basic theories have been proposed. First, these changes may result from a rise in intracranial pressure (ICP) from cephalad fluid shifts during LDSF. A second possible explanation for SANS is compartmentalization of cerebrospinal fluid (CSF) within the orbital optic nerve sheath. In the past, it was generally assumed that there was homogeneity of ICP and chemical components of the CSF throughout the brain, spinal cord, and orbital optic nerve sheath. However, the unique tightly confined and cul-de-sac like anatomic connection between brain and orbit in the optic nerve sheath may create a fragile flow equilibrium and a possible one-way valve like effect that may lead to pressure elevation within the orbital optic nerve sheath with or without elevated CSF pressures surrounding the brain. It has also been proposed that a microgravity induced glymphatic flow imbalance within the orbit may play a role in this process.

These potential etiologies and pathogenesis for SANS are inadequate for describing the objective measures of no to mild increase in the in CSF during space flight, normal or near normal autoregulation of cerebral blood flow, and normal brain imaging.

In view of the foregoing, this invention provides a unique mechanism of SANS, a mechanism of measuring the predisposing to developing SANS prior to space flight and measuring the development of SANS, a mechanism of monitoring for the efficacy of countermeasures, and a mechanism for detecting ongoing injury during space flight.

In one aspect, this invention discloses a novel method that uses ultrasound imaging to measure the predisposing factors for the diagnosis of SANS and measurement of the extent of the risk of developing SANS on the ground and in a microgravity environment, and to monitor in the microgravity environment the effectiveness of SANS countermeasures or treatments. The novel method is also used to detect on going ocular injury in a microgravity environment as well as for resolution of SANS predisposing factors once returning to the Earth.

The optic nerve sheath (ONS) is a structure surrounding the optic nerve as it exits the skull and contains CSF. Elevations in the ICP causes an increase in the ONS with a proportional increase in the ONS pressure. In addition, the diameter of the ONS may be a marker that characterizes pathological increases in the ICP.

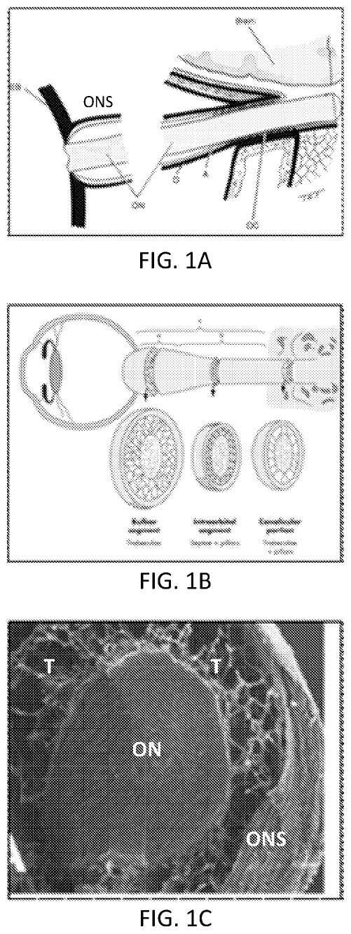

Structurally, the ONS is attached to the optic nerve via trabeculae, septae, and pillars, as shown in FIGS. 1A-1C. In a normal state, as also called herein a “competent” ONS, these structural components of the ONS resist the expansion or dilation of the ONS as the CSF undergoes physiological pressure fluctuations from the cardiac cycle, respiration, body position and physical activities. In the competent ONS, the diameter, size and volume do not change with small changes in the CSF pressure that do not exceed the structural component strength to resist the pressure.

However, throughout life, events occur that cause the ICP to exceed the structural resistance or integrity of the ONS components. Classically moderate and severe traumatic brain injury (TBI) may cause enough of an elevation in the ICP to damage the ONS structural components. Other conditions such as mild traumatic brain injury (mTBI), diseases such as pseudo tumor cerebri, meningitis, severe hypertension, or lifestyle activities such as extreme training or weightlifting can result in an ICP elevation large enough to damage the ONS structural support. Once these structures are damaged from an “elevated ICP event”, they are no longer capable to resist minor changes in the ICP or intracranial volume. The ONS size and volume increases with small, normal physiologic variations in ICP. The ONS size and volume can increase with small changes in the ICP that are out of proportion to what would be expected with a competent ONS with activities that increase intracranial volume such as a head down position or Valsalva, which is referred herein as an “incompetent” ONS. There are varying degrees of the incompetent ONS, based on the extent (severity) and duration of the elevated ICP event and the degree of incompetence can be demonstrated by the degree of dilation to physiologic changes in ICP or changes in volume due to activity.

As shown FIGS. 1A-1C, the ONS surrounds the optic nerve (ON) and contains cerebrospinal fluid (CSF) (FIG. 1A). The ONS is connected to the ON by structural components called trabecula, septa, and pillars (FIG. 1B). FIG. 1C shows a scanning electron microscopy image of the ON, trabecula (T) and ONS. Because these components (FIG. 1C) expand at different rates with changes in the ICP, the 3-dimensional (3D) shape of the ONS can be used to detect subtle changes in the ICP. The structural components of the ONS (trabecula, septa, pillars) can be damaged by a variety of activities and injuries. When they are damaged, they lose the ability to bind the ONS to the ON.

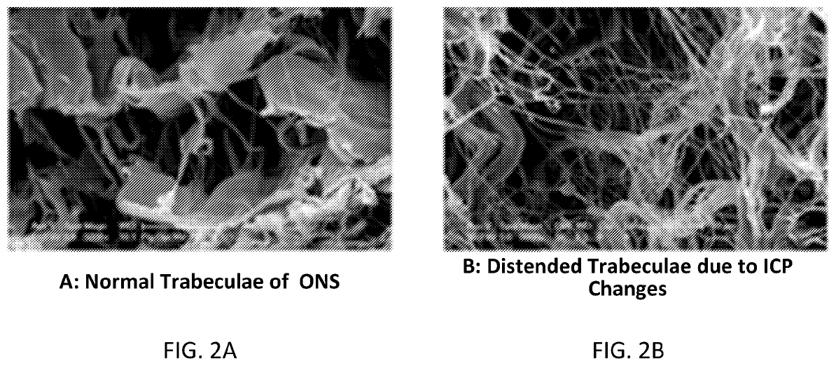

Since the structural components (trabeculae, septae, and pillars) as well as the individual fibers themselves are of varying density and structural strength, they react differently to pressure in an elevated ICP event. During the elevated ICP event the fibers become stretched or broken. Once the fibers are stretched or broken, they lose structural integrity. The trabeculae can be damaged by a variety of mechanisms including an episode of elevated intracranial pressure. In one embodiment, as demonstrated in a porcine animal model, after a single ICP elevation episode, the trabeculae of the ONS become thinned and frayed (FIG. 2B), correlating the dilation in the ONS diameter to the loss of structural integrity in the trabeculae. FIGS. 2A-2B show scanning electron microscopy images of the trabecula of the ONS, with the normal appearance of the trabecula (FIG. 2A) and the trabecula after damage (FIG. 2B). In this case, an increased intracranial pressure is the cause of the damage. Normally, the ONS is tightly bound to the ON and the 3D surface structure of the ONS is uniform and homogenous. When the underlying structure (trabecula, septa, pillars) are damaged, this damage can be visualized by visualizing the surface of the ONS using 3D ultrasound. The greater the elevation and duration in the ICP during the elevated ICP event, the more extensive the damage to the trabeculae. The more the damage, the more irregular the surface of the ONS.

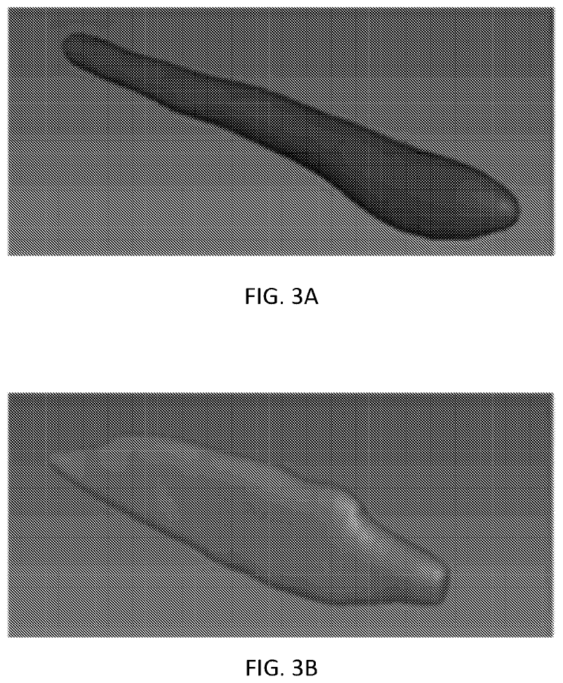

The 3D structure of the competent ONS is fairly uniform in size, usually oval to round with smooth sides as the structural components that form this shape are not damaged. As the ICP exceeds the structural capacity of the individual fibers of the ONS, there is a loss of the structural integrity of the individual trabecular components resulting in an alteration from the normal shape with the deviation from the normal shape at the location of the trabecular damage. As noted above, the greater the elevation and duration, the more widespread the trabecular damage is. By using 3D ultrasound imaging of the ONS surface structure, the bulging out of the sheath due to the loss of the structural integrity of the ONS trabeculae can be visualized. Since the elevated ICP affects the weakest components first, as the pressure increases, there is a bulging out of the smooth surface of the ONS at localized areas. As the pressure continues to increase, there are more fibers that break or stretch resulting in more and more bulges in the ONS surface structure. At the maximum extent of the ONS (loss of all trabeculae structural integrity), all the fibers are stretched or broken, having lost all structural integrity, and the ONS resumes a more “normal” appearance of a smooth surface but the overall size and volume of the sheath has increased due to the excessive CSF pressure, as shown in FIG. 3B, which demonstrates the loss of structural integrity of the ONS trabeculae in a non-uniform fashion causing bulging on the sides of the ONS indicating the loss of structural integrity in localized areas of the ONS in a porcine model. As the pressure increases, there are increasing areas of bulging of the ONS.

Both the peak pressure of the ICP elevation and the duration of pressure elevation are important in the pathophysiology of the ONS structure. ICP pressure increases can be short lived or instantaneous to very long lasting depending on the reason for the elevated ICP. The duration of the ICP elevation is nearly as important as the absolute elevation in the ICP. Both affect the amount or extent of trabecular damage, the loss of the ONS structural integrity, and resultant bulges in the surface structure of the ONS. Further damage to the ONS structural integrity can be constituted by further, or recurrent, increases in ICP.

Over time, the ICP will decrease (unless the person dies). As the ICP decreases due to disease progression or due to therapy, the ONS pressure decreases proportionally with the decrease in the ICP. While the pressure in the ONS decreases concomitant with the decrease in ICP, the ONS structure will remain in its dilated state. This is because pressure and volume in the ONS are no longer coupled. This had been demonstrated by the inventor in both cadaveric and porcine models of the elevated ICP.

In a competent ONS, the pressure in the sheath, the ICP and the ONS volume are all related, increasing or decreasing simultaneously and proportionally. In an incompetent sheath, as the structural components of the ONS are damaged after an elevated ICP event, while the pressure is decreasing, the 3D surface structure does not change for some time as the ONS pressure and volume are no longer correlated. Decreases in the ICP and ONS pressure are not accompanied by a decrease in volume of fluid as would be in the competent ONS.

The damaged fibers are not able to cause a pressure gradient to squeeze out or decrease the volume of CSF that accompanied the increase in CSF pressure. Hence the size and surface structure of the ONS does not decrease concomitantly with the decrease in the ONS pressure and ICP decrease. There is a delay in the fluid volume draining out of the ONS. The delay in the volume decreasing and the reduction in size is related to the extent of the damage to the structural components of the ONS-the degree of incompetency. Even after the volume has decreased to a normal baseline level, because the underlying ONS structures (trabeculae) are damaged, the ONS will not return to its pre-injured baseline shape. The overall size may return to “normal” or near baseline size, but the surface structure will demonstrate the underlying trabecular damage. The underlying trabecula damage can be visualized and measured using 3D ultrasound imaging of the ONS surface structure where the normal smooth surface is now wrinkled or bulged. As demonstrated in FIGS. 3A-3B showing ultrasound imaging of the ONS in an experimental porcine model, where FIG. 3A is the baseline appearance of the ONS showing a smooth surface and FIG. 3B is a damaged ONS of the same individual. The damage in this case is caused by an elevation in the ICP. The bulges or wrinkles are proportional to the increase in the ICP as the structural resistance of the individual trabeculae are exceeded by the pressure.

With subsequent elevations in the ICP that exceed the newly established structural limit of the trabecula, the bulges become more apparent or dramatic as more and more trabecula are broken or stretched. The trabecular structural damage appears to be permanent. In the porcine experiment, the structural damage observed in the trabecula in animals euthanized immediately after the increased ICP event and in those allowed to live for 30 days were the same.

Using a provocative maneuver and 3D ultrasound surface imaging, the structural damage can be further evaluated. A provocative maneuver is one that increases the volume in the ONS, causing it to dilate with minimal increases in ICP. This dilation is demonstrated in retrospective and prospective human studies using a Valsalva maneuver as a provocative maneuver.

The Valsalva maneuver (increasing intrathoracic pressure) decreases the venous drainage from the head and very mildly increases the ICP. As the drainage from the cranium is decreased, fluid accumulates in the ONS. In a competent ONS, as the volume tries to increase, the structural components of the ONS resist dilation. In a damaged or incompetent ONS, the ONS is structurally weakened and is unable to resist the volume. With 3D ultrasound imaging of the ONS, the surface of the ONS begins to fill in the wrinkles, increasing in overall size. This is different than in an initial elevated ICP event that is damaging the ONS structures. Here, the ICP and ONS pressure during the Valsalva maneuver are not increasing as it does in the elevated ICP event. What is happening is that the volume in the ONS is increasing, distending the weakened sheath. The dilation during the Valsalva maneuver (or any activity that decreases venous drainage from the cranium such as a head down position) is due to the volume. With a prolonged Valsalva maneuver, the sheath approximates the shape and size from the elevated ICP event after enough volume has accumulated in the damaged ONS. This relationship had been demonstrated by the inventor in a retrospective study, which demonstrated that individuals with a remote history (>1 year) of mild traumatic brain injury (mTBI) (elevated ICP event) showed significant dilation of the ONS after a Valsalva maneuver, while those without a history of mTBI did not dilate.

In addition, the causal relationship between the occurrence of a new mTBI (ICP increasing event) and the ONS dilation following a Valsalva maneuver is also demonstrated by the inventor in a prospectively study, where a cohort of mixed martial art (MMA) fighters was prospectively followed. Individuals with the competent ONS (did not dilate with the Valsalva maneuver) and no history of TBI, concussion or other history of an elevated ICP event were selected. The individuals were followed throughout the year during MMA fights for assessing the ONS and for a concussion or mild TBI. Three individuals suffered a mild TBI or elevated ICP event, documented by a ring-side doctor. On follow up of the fighters who sustained the mTBI exhibited a post-Valsalva dilation of the ONS. Further, these individuals continued to dilate with the Valsalva maneuver for the remainder of the study (one year). Individuals who did not have an elevated ICP event during the study showed no change in the ONS dilation post-Valsalva. These results were consistent with our prior retrospective study.

These baseline studies and their results are relevant to the application for SANS. The important points are that the ONS, once damaged by even transient increases in ICP, is structurally changed. With additional ICP events, there is increasing damage to the ONS structural integrity. With a Valsalva maneuver, the pressure in the ONS minimally changes but the volume increases the size of the ONS to resemble the post-elevated ICP state.

To describe the pathology of SANS, this accumulation of fluid in the ONS is important. In normal gravity situations, a damaged ONS does not impact the ON or visual function as gravity prevents the ONS from retaining CSF fluid. In other words, gravity aids the drainage of the incompetent ONS, not depending on the structural integrity of the ONS to prevent accumulation of CSF in the sheath.

In a microgravity environment, gravity no longer aids in the drainage of CSF from the ONS. This results in volume accumulation in the ONS, similar to performing a Valsalva maneuver. In a competent ONS, there is a minimal accumulation of CSF volume. In individuals with an incompetent ONS, the volume is increased in the ONS. To an incompetent ONS, a microgravity environment is similar to performing a prolonged (duration of space flight) Valsalva maneuver. Since this is an abnormal volume of fluid, with impaired drainage, there is a variety of impacts to both the flow of the CSF in the ONS as well as to the microscopic structures of the ON and retina leading to the constellation of symptoms known as SANS.

Importantly, the ONS houses the blood vessels which supply the optic nerve and retina, primarily, via the posterior ciliary arteries and the central retina artery and vein. While retinal vessels lack fenestration and thus have blood-brain barrier properties, branches of the posterior ciliary arteries may or may not be fenestrated. Retina and optic nerve head astrocytes housed within these structures surround these blood vessels and are thought to contribute to the regulation of blood flow.

In SANS, due to the microgravity environment, the incompetent ONS leads to increased volume (CSF) leading to elevated sheer stresses on the venous endothelial cells within the posterior laminar region due to the stretching associated with the volume. The increased volume leads to local endothelial cell shear and dysfunction at the cellular level, causing the vascular function of the microscopic vessels to be altered. When the function is altered in these microscopic vessels, there is a wide variety of manifestations, including localized ischemia, increased vascular permeability, and loss of the blood brain barrier.

Blood flow to the brain, including the eye, is regulated by multiple factors that establish a longitudinal pressure gradient; these factors can be categorized into three main components: cardiovascular, intracranial pressure (ICP), and cerebrovascular. Understanding the relationship among these systems is pivotal to understanding ocular/cerebral blood flow (CBF) regulation. Importantly, the impact these components have on the local microcirculation is ill-defined but critically linked to neuronal and ocular function. Simplified, CBF is dependent on the pressure supplied by the cerebral arteries (ABP), the backpressure in the cerebral venous system (similar to ICP), and small vessel resistance (cerebrovascular resistance (CVR)). The importance of this relationship, defined as CBF=(ABP−ICP)/CVR), lies in the strong interdependence of the physiological variables that establish CBF. Under normal conditions, CBF is regulated by cerebroprotective mechanisms (e.g., cerebral autoregulation, baroreceptor sensitivity). At the vascular level, CBF is controlled by dynamic changes in vessel diameter, which in turn influence CVR. Whereas arterioles play a major role in the active regulation of CBF, larger vessels and capillaries are also involved. Although it has long been suggested that the high degree of compliance of cerebral venules and veins plays a passive role in the regulation of CBF and/or pressure gradients, a recent reconceptualization has challenged this assumption by proposing instead that external pressures acting on these vessels can evoke a Starling resistor-like effect. These external pressures, which here include that of the optic nerve sheath microenvironment, are of particular importance in a weightless environment where cephalad fluid shifts contribute to constitutive higher than normal ICP and altered pressure gradients. In individuals with an incompetent ONS, the added volume can increase the capillary and venous pressures in the eye/brain. In the healthy brain, changes in CVR or CBF can occur as a result of neuronal activity (e.g., neurovascular coupling) and changes in vessel tone (i.e., dilation/constriction). Together with ventilation-related changes in the partial pressure of carbon dioxide (PCO₂)—evoked reactivity, autonomic activity and neuronal activity, the complex interdependence of pressure variations makes it challenging to unravel the physiological and cellular mechanisms that determine blood flow to the brain.

To maintain optimal function, the brain/eye requires uninterrupted blood flow. One of the hallmarks of neurodegenerative disorders, and a contributing factor to cognitive decline, are decreases in CBF (ischemia). Elevated intravascular pressure (i.e., hypertension), promotes cerebrovascular dysregulation, inflammation, decreased blood-brain barrier integrity, impaired toxic/metabolic waste clearance (glymphatic function), and small vessel disease. In SANS we postulate that elevated extravascular volume and localized pressure from the increased volume lead to similar pathologies. Components of the neurovascular unit, which at a minimum includes neurons, astrocytes, endothelial cells, and vascular smooth muscle cells or pericytes, are important in these processes, implying that altered pressure gradients intersect with the neurovascular unit to exert its effects.

Localized pressure gradients in the ONS can have similar pathological effects to that seen in the elevated ICP. Enlargement of the optic nerve subarachnoid space may lead to loculation of the CSF within the space, increasing the pressure in the ONS without an increase in the ICP. The reduced CSF flow leads to stagnation and reduced removal of toxic metabolites and reduced delivery of necessary metabolites within the anterior, posterior and retrolaminar regions of the optic nerve.

This stagnation may have an important effect on the function of lymphatic and neuronal tissues of the ONS, ON, and retina. The local volume increase associated with the localized pressure increase within the ONS is associated with compression of the central retinal and other veins, leading to reduced anterior optic nerve perfusion leading to further damage to the ganglion cell axons. The altered CSF outflow combined with a microgravity environment-induced increase in the CSF volume that is not reduced by postural changes may be important contributors of the SANS.

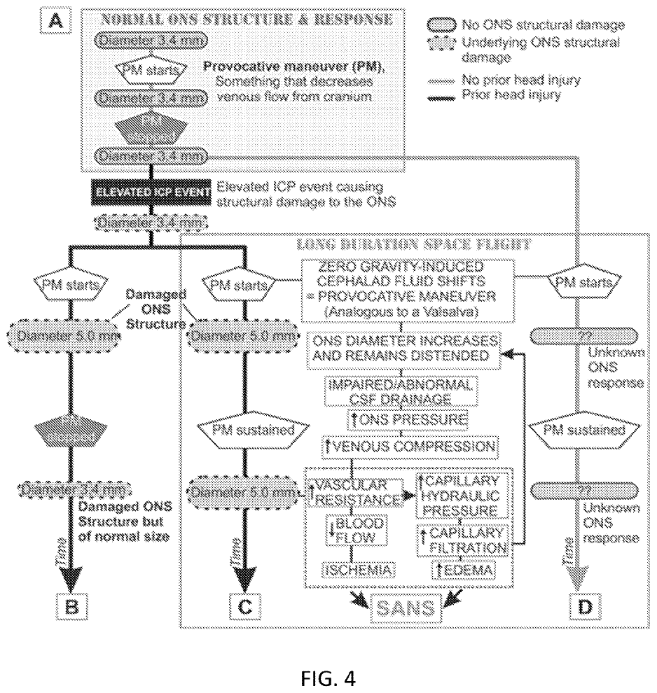

As discussed above, the structural changes in the ONS are a key component in the development of SANS symptoms. In space, the lack of gravity causes a cephalic shift in fluid, yielding head and neck congestion and is similar to performing a Valsalva maneuver. Since astronauts are on the International Space Station (ISS) for long periods, this environment mimics the physiology of the Valsalva maneuver but continuously for months on end. Under these conditions, the ONS does not have the chance to recover or return to its baseline size. Our preliminary data provide a hypothesis of the underlying basis for the SANS. We postulate that microgravity-induced fluid shifts alter the hydrodynamics of the CSF in the ONS, which, due to its altered integrity, now allows CSF to amass and distend within the sheath, causing a local increase in the intra-sheath pressure (FIG. 4). The intra-sheath pressure elevation is distinctly different from ICP elevation, though the intra-sheath pressure rises as the ICP rises but not vice-versa. The elevated intra-sheath pressure causes the capillary hydrostatic pressure to increase in the retina and the ONS, where the blood vessels to the optic nerve and retina course. Increased capillary hydrostatic pressure favors vascular leakage into the interstitial space of the optic nerve head and subretinal space (as demonstrated on retinal imaging in astronauts with the SANS). In addition, altered pressure gradients increase vascular resistance, via increased venous compression, exacerbating the hydrostatic pressure-evoked edema. In this case, while mean systemic arterial blood pressure may stay within normal ranges, the intravascular pressure of vessels coursing along and through the ONS is subjected to higher wall tensions increasing vascular resistance and, consequently, reduce blood flow to the distal tissue of the optic nerve and retina. This sequence of events may explain a previously overlooked mechanism leading to the ocular pathology, optic disk edema, choroidal retinal folds, and nerve fiber layer infarcts, characteristic of the SANS, which in turn is similar to that observed with severe systemic hypertension. While SANS is multifactorial, this mechanism, determined by the variability in the structural integrity of the ONS, explains the findings that only some of the astronauts develop the SANS, and others do not. Each astronaut has an individual variability in the ONS response to a low or no-G environment based on their previous history of elevated ICP events, similar to the groups of individuals we described in our retrospective and prospective studies.

FIG. 4 shows schematically influence of microgravity on the dynamic ONS structural and functional alterations before and after head injury. A. Normal conditions, a provocative maneuver (PM), such as a Valsalva maneuver, has no significant effect on the ONS size. Consequently, at the end of the PM, the ONS diameter is comparable to normal. B. In individuals with a history of prior head injury, an elevated ICP event causes structural damage to the ONS. Here, in response to a PM, the ONS cannot resist the change in volume and dilates. Once the PM is stopped, the ONS returns to its baseline size. In space, the microgravity is the PM, simulating a continuous Valsalva. C. In an individual with a damaged ONS, microgravity/PM will exaggerate the volume in the ONS due to the lax structure. The resulting increase in local pressure compresses venules, increase vascular resistance and capillary hydraulic pressure affecting both blood flow and capillary permeability in the optic nerve and retina. These deleterious events compromise the optic nerve and retinal function. The sustained ONS diameter distension establishes a positive feedback loop which, with time, exacerbates SANS pathology and symptoms. D. In an individual with no prior head injury, microgravity/PM and the lack of lax ONS structure could either worsen or protect the optic nerve and retina.

This mechanism of the SANS can be evaluated using 3D ultrasound imaging of the ONS combined with elastography of the ONS walls. As disclosed above, we discussed how the ONS structural integrity can be measured using 3D US imaging of the surface structure, combined with a provocative maneuver such as a Valsalva maneuver. According to the invention, this is a diagnostic and prognostic indicator that can be utilized in the evaluation of the SANS. Since trabecular integrity is inversely related to the ONS volume accumulation in a microgravity environment, evaluation of these properties can satisfactorily be accomplished using this method.

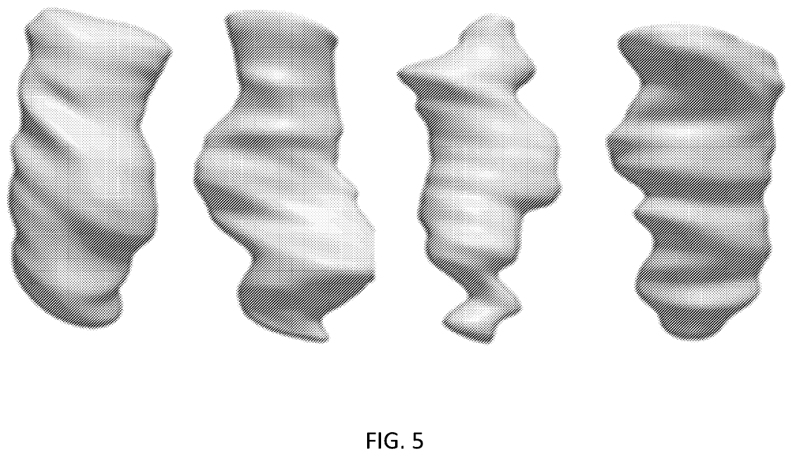

FIG. 5 shows 3D ultrasound images of a human with a severely damaged ONS. The severity of the damage is proportional to the degree of the irregularity of the surface structure seen in the image. The more variability of the surface, the more underlying structural damage and the higher the risk of developing SANS in a low gravity environment.

This variability can be objectively measured in many ways: 1) mean radius at a specified point in the ONS distal to the retina and the standard deviation in the radii, 2) volume at a specific level measured posterior to the retina (for example the ONS volume 2 mm to 4 mm posterior to the retina, 3) volume of the ONS compared to an ideal cylinder. All these measurements require a pre-measurement compared to a post Valsalva measurement. The difference between the pre-measurement and the post Valsalva measurement is proportional to the ONS structural damage with the absolute measures given in any of the methods above. For example: the pre-Valsalva volume is 0.157 ml, and the post Valsalva volume is 0.211 ml. This difference in volume of 0.054 ml is consistent with mild ONS damage and the risk of SANS is unlikely with prolonged exposure to a low gravity environment.

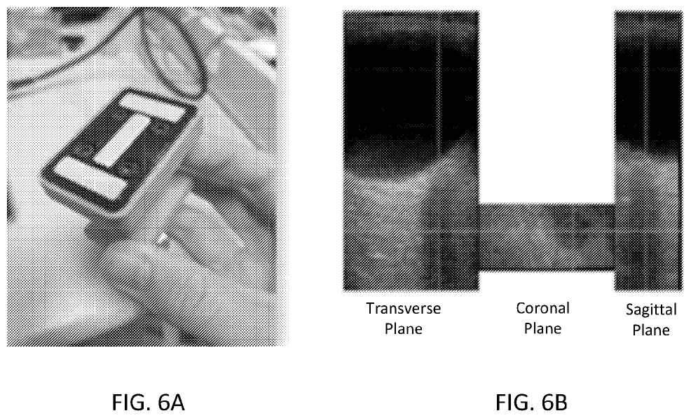

In some embodiments, the process uses a specially designed 2D ultrasound to obtain purpose-directed ultrasound images of the ONS. Due to the design, we can create a 3D volume of the ONS can be created and used to detect subtle changes in the ONS surface, which can be correlated to the ONS structural damage (and severity) and then to the likelihood of developing SANS. FIG. 6A shows one embodiment of a specially designed bedside/portable ultrasound device using 3 linear arrays to simultaneously image the ONS allowing for reconstruction of the 3D volume. Using the 3D volume of the ONS, we create 3D images of the ONS and/or a quantitative measurement of the structural damage and volume of the ONS, as shown in FIG. 6B.

As demonstrated in a gravity environment, our method can be used to detect baseline damage the ONS. Using a Valsalva maneuver, the fluid shift of a zero or low gravity environment such as in outer space flight can be replicated. According to embodiments of the invention, a Valsalva maneuver can be used to measure the structural integrity of the ONS and the susceptibility to SANS when in a zero or low gravity environment.

In addition, the stiffness of the ONS can be correlated to the pressure of the CSF in the ONS. To do this, a specially designed portable ultrasound is used with elastography mode. Elastography is an ultrasound mode that evaluates tissue stiffness. Elastography gives a measurement of the deformation of the tissues from the sound wave pressure produced with ultrasound imaging. Elastography is a key component of this method of evaluating for SANS. Elastography gives a mechanism to measure the pressure on the ONS wall. As described above, the ONS can expand with either pressure (competent or incompetent ONS structural integrity) or with volume (incompetent only). Elastography allows for determination if the dilation is due to the ONS being due to pressure or volume. In a competent ONS, the elastography demonstrates increasing wall stiffness as the pressure increases. Similarly in an incompetent ONS, elastography demonstrates increasing wall stiffness as the ONS pressure increases. In contrast with an incompetent ONS, dilation due to volume, without a pressure increase, such as in a microgravity environment, will remain normal (no increase in ONS wall stiffness).

Using the 3D surface structure, elastography and provocative maneuvers, the ONS of an individual can be assessed before being subjected to a microgravity environment to measure the amount of ONS structural damage. The greater the damage presents, the more likely the individual will develop SANS in a microgravity environment. This risk assessment will aid in assessing the likelihood of developing SANS allowing for planning of shorter exposure to a microgravity environment or to allow for mitigation or counter measure therapies to be instituted during the microgravity exposure.

Elastography demonstrates the stiffness of the ONS wall, correlating to the pressure of the fluid in the ONS. Combining the ONS surface structure or 3D derived measurements in combination with and without a Valsalva maneuver allows us to determine the pressure on the ONS and retina. Using the change in these values we can real time detect the onset and monitor for the development of SANS and also measure the effectiveness of any treatment or preventative measures. In other words, the pressure and the volume can be measured sequentially during outer space flight with and without a SANS intervention for prevention or treatment.

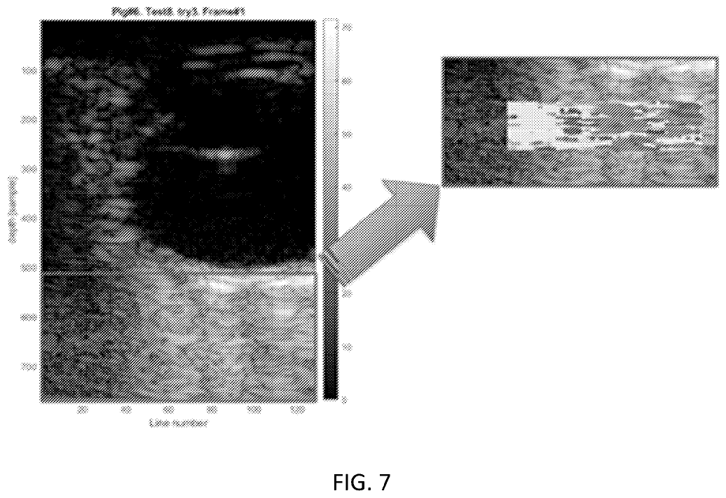

FIG. 7 is a real-time elastography of the ONS. The colors are qualitative indicators (quantitative measurements are reported in kPa) of the stiffness and consequentially the amount of pressure on the optic nerve sheath. The qualitative colors are used to indicate there is a pressure on the ONS and there should be increased monitoring for ONS damage. The qualitative measurement is used to give the exact pressure on the ONS with a higher pressure indicating that there is ongoing potential for ONS damage and the rate of the damage. Conversely, the qualitative and quantitative measurement of the ONS can be used to determine the effectiveness of SANS treatment or countermeasures or be used to adjust (tailor) the treatment to the individual.

While in a microgravity environment, 3D surface structure, elastography and provocative maneuvers provides a mechanism to determine the amount of volume accumulation in the ONS. The ONS can be monitored to evaluate if there is accumulating damage, increasing the likelihood of developing SANS or increasing the severity of SANS or to determine if there is a concomitant increase in ONS pressure. Measuring these variables serially with the initiation of counter measures (treatments) for SANS, will give an assessment of the effectiveness of the counter measure (treatment). This would allow for adjustment of the counter measure to the individual, increasing its effectiveness.

Once the individual returns to the Earth, the ONS can be assessed for damage due to the microgravity environment. This will give an indication as to the long-term implications of the microgravity exposure, aiding in determining if there is a life-time limit that should be set to prevent SANS development in a corps of astronauts.

Low gravity Experiment: Using noninvasive 3D ultrasound imaging of the ONS, we can give a comprehensive evaluation of the preflight, flight, and postflight ONS structure, pressures, and volumes, allowing for a determination of susceptibility, time course, severity of SANS and the effectiveness of treatments meant to mitigate or prevent the development of SANS during space flight. As part of low gravity parabolic flights, the mechanics of the methods have been proven. Astronauts can perform the measurements in a low gravity environment.

Methods: This is a cross sectional observational study. Study team members trained in 3D ultrasound measurement acquisition obtain baseline images at rest and with Valsalva prior to flight, daily (FD1, FD4, FD5) in a micro-gravity environment and after return to gravity.

In some embodiments, the participants are sorted into groups with damaged and undamaged ONS at baseline before flight. This was done using the technique above and a Valsalva maneuver. The amount of damage in the ONS is graded in the damaged ONS cohort.

In some embodiments, the volume accumulation in space flight (low gravity) in the damaged versus the undamaged cohorts of the ONS is measured. The elastography (stiffness/pressure) associated with the volume accumulation is also measured. Once on the ground (normal gravity), the surface structure is measured for assessing accumulated damage (from baseline) correlated to the extent from the volume and/or pressure changes within and between the two cohorts.

In some embodiments, future flights assess the ability to evaluate the ability to measure SANS countermeasures while in a low gravity environment.

As disclosed above, the SANS is due to increased volume of CSF in the ONS. Elevated intracranial events lead to damage to the ONS structure, specifically the trabecula that hold the optic nerve sheath to the nerve. Damage is long-lasting, possibly permanent, and is proportional to the rise in the ICP at the time of the event and is cumulative with additional elevated intracranial events. Once the damage occurs, the ONS dilates with decreases in venous drainage from the head, i.e., “provocative maneuvers”. Normally, the ONS does not dilate without an increase in ICP. However, a damaged ONS dilates with the increase in volume without (or with a mild) increase in ICP. A ONS that dilates with volume without an increase in ICP is referred herein as an incompetent ONS. Provocative maneuvers include anything that decreases blood flow from the cranium, such as a Valsalva maneuver, a head down position, weightlifting, etc.

In some embodiments, the increase in volume in the ONS causes local pressure effects on the retina and optic nerve. This increase in pressure leads to retinal changes seen in the SANS.

In some embodiments, the dilation of the ONS leads to an alteration in CSF flow in the ONS. This leads to a decrease in circulation of the CSF around the nerve.

In some embodiments, prolonged increases in the volume in the incompetent ONS leads to further trabecular damage, increasing the ONS volume over time.

In some embodiments, there is a threshold at which the volume causes pathology. The amount of damage to the ONS structure (trabecula) is the factor in determining the volume in the ONS.

In some embodiments, 3D ultrasound is used to measure and visualize the ONS surface structure. The ONS surface structure is representative of the underlying trabecular structure.

In some embodiments, using a provocative maneuver, the 3D surface structure can be used to determine the extent of underlying trabecular damage and ONS incompetency.

In some embodiments, in a microgravity environment, the 3D surface structure can be used to measure the change in volume in the ONS. It can also be used to measure changes in the ONS volume due to countermeasures or due to ongoing microgravity changes.

In some embodiments, elastography is used to measure the tension on the wall of the ONS. When the volume in the ONS is increased but the ONS pressure or ICP is not elevated, the elastography be normal, consistent with the tissue surrounding the ONS and across the optic nerve. As the ICP increases with an increasing volume, the elastography demonstrates a stiffening ONS. With increases in ONS pressure, the 3-dimensional surface structure can be monitored to detect ongoing structural damage.

In some embodiments, the method can determine if an individual is at risk of developing SANS by evaluation of the ONS integrity using bedside/portable ultrasound creating a 3D image or 3D derived quantitative measurement of the ONS.

In some embodiments, the method can evaluate or grade the risk of an individual developing SANS by determining the extent of the damage to the ONS integrity using bedside/portable ultrasound creating a 3D image or 3D derived quantitative measurement of the ONS and a Valsalva maneuver. Grading includes a relative propensity of developing SANS based on exposure to a low gravity environment. Grading correlates the “dose” of low gravity to the risk of developing SANS. This could be in an estimated risk versus duration, or an estimated safe flight duration (length in time that exposure of low gravity would be safe).

In some embodiments, the method can real-time monitor for ONS damage due to astronaut and other training by measuring the ONS integrity over time and comparing to individual baseline using bedside/portable ultrasound creating a 3D image or 3D derived quantitative measurement of the ONS and a Valsalva maneuver.

In some embodiments, the method can real-time monitor for the development of SANS while in a zero or low gravity environment such as in space using a bedside/portable ultrasound creating a 3D image or 3D derived quantitative measurement of the ONS.

In some embodiments, the method can real-time monitor for increased optic nerve and retinal pressure due to an incompetent ONS structure using a bedside/portable ultrasound creating a 3D image or 3D derived quantitative measurement of the ONS and elastography of the ONS.

In some embodiments, the method can real-time monitor for the effectiveness of a SANS treatment or prevention while in a zero or low gravity environment (outer space) using a bedside/portable ultrasound creating a 3D image or 3D derived quantitative measurement of the ONS and elastography.

In some embodiments, the method can determine the effect of space flight on the individual after the flight by determining the accumulated ONS damage from spaceflight using a bedside/portable ultrasound creating a 3D image or 3D derived quantitative measurement of the ONS.

In some embodiments, the method can determine when the individual has returned to their baseline ONS volume and pressure after flight using a bedside/portable ultrasound creating a 3D image or 3D derived quantitative measurement of the ONS and elastography.

In some embodiments, by detecting the subtle changes in the ONS surface structure using 3D ultrasound and/or 3D quantitative measurements, correlating the ONS surface structure and surface changes from baseline/normal to the underlying structural components of the ONS connections to the ON, and grading the structural integrity, an amount of damage to the ONS structure and consequently the loss of structural integrity can be determined or graded.

Without intent to limit the scope of the invention, some exemplary embodiments are further given below:

In some embodiments, the method for screening SANS comprises obtaining 3D images or 3D quantitative measurements of at least one ONS of the living subject at rest and with a provocative maneuver, respectively, prior to a spaceflight, wherein the 3D images or the 3D quantitative measurements obtained at rest is used as a baseline; evaluating the 3D images or the 3D quantitative measurements obtained at rest and with the provocative maneuver; and determining if the living subject is at risk of developing the SANS based on the evaluation of the 3D images or the 3D quantitative measurements.

In some embodiments, the provocative maneuver is an activity that increases the volume in the ONS, causing the ONS to dilate with minimal increases in the ICP.

In some embodiments, the provocative maneuver is a Valsalva maneuver.

In some embodiments, the 3D images are 3D ultrasound images obtained by an ultrasound probe, and wherein the 3D quantitative measurements are obtained by a magnetic resonance imaging (MRI) or computed tomography (CT) scanner.

In some embodiments, said evaluating the 3D images or the 3D derived quantitative measurements comprises evaluating a structural integrity of the ONS using the 3D images or the 3D quantitative measurements obtained with the provocative maneuver, by comparing them with the baseline, wherein the structural integrity is a diagnostic and prognostic indicator utilized in the evaluation of the SANS.

In some embodiments, said evaluating the structural integrity of the ONS comprises obtaining changes in a surface area of the ONS by comparing the 3D images or the 3D derived quantitative measurements obtained with the provocative maneuver with the baseline.

In some embodiments, said evaluating the structural integrity of the ONS comprises obtaining changes in a volume of the ONS by comparing the 3D images or the 3D derived quantitative measurements obtained with the provocative maneuver with the baseline.

In some embodiments, said evaluating the structural integrity of the ONS comprises obtaining changes in a surface structure of the ONS by comparing the 3D images or the 3D derived quantitative measurements obtained with the provocative maneuver with the baseline.

In some embodiments, said obtaining the changes in the surface structure of the ONS comprises obtaining a degree of irregularity of the surface structure of the ONS visualized in the 3D images or the 3D derived quantitative measurements obtained with the provocative maneuver. It should be noted that used herein, the term “degree of irregularity” of the surface structure may refer to the presence of and degree of surface irregularity as defined by as a range of variations per unit along the surface of the optic nerve sheath outer surface, or alternatively, to a degree of ONS surface irregularity as defined by the roughness parameters (3D derived quantitative measures such as the average radius from the center of the ONS to the surface, the radius standard deviation, radius minimum and maximum) or the irregularity of the ONS surface through visualization of the 3D image (qualitative).

In some embodiments, the irregularity of the surface structure of the ONS includes bulges, wrinkles, and/or ripples formed on the exterior surface of the ONS.

In some embodiments, the degree of irregularity of the surface structure of the ONS is associated with a severity of a structural damage of the ONS, wherein the severity of the structural damage is proportional to the degree of the irregularity of the surface structure, and the more variability of the surface structure of the ONS, the more underlying structural damage and the higher the risk of developing the SANS in a low-or no-gravity environment.

In some embodiments, the variability of the surface structure of the ONS is measured by one or more of a mean radius at a specified point in the ONS distal to the retina and a standard deviation in the radii; a volume at a specific level measured posterior to the retina; and a volume of the ONS compared to an ideal cylinder. Such a comparison to an ideal cylinder can give the 3D concept that there is “lumpy bumpy” structure to the ONS both in the cross section (circle) view and along the length of the sheath. That is, the more “lumpy bumpy” (or the rougher the surface) the worse the underlying damage (less competent) the sheath and the more likely to develop SANS.

In some embodiments, the 3D images or the 3D quantitative measurements are of real-time elastography of the ONS, wherein said evaluating the 3D images or the 3D quantitative measurements comprises obtaining changes in a pressure of fluid in the ONS using the 3D images or the 3D quantitative measurements.

In some embodiments, said determining if the living subject is at risk of developing the SANS comprises determining a degree of predisposition to developing the SANS based on the structural integrity of the ONS.

In some embodiments, the degree of predisposition to developing the SANS includes a relative propensity of developing the SANS based on exposure to a low-or zero-gravity environment.

In some embodiments, said determining the degree of predisposition to developing the SANS comprises correlating a dose of low gravity and/or a duration of exposing to a low-or zero-gravity environment to the risk of developing the SANS.

In some embodiments, the method further comprises obtaining 3D images or 3D quantitative measurements of the ONS of the living subject during the spaceflight and/or on a space station in a low-or no-gravity environment; evaluating the 3D image or the 3D quantitative measurement obtained during the spaceflight and/or on the space station in the low-or no-gravity environment by comparing them with the baseline; and determining a development of the SANS, an efficacy of SANS treatments or countermeasures, and/or an ongoing injury during the spaceflight and/or on the space station in the low-or no-gravity environment based on the evaluation of the 3D images or the 3D quantitative measurements.

In some embodiments, said evaluating the 3D images or the 3D derived quantitative measurements comprises evaluating a structural integrity of the ONS using the 3D images or the 3D quantitative measurements obtained during the spaceflight and/or on the space station in the low-or no-gravity environment, by comparing them with the baseline, wherein the structural integrity is a diagnostic and prognostic indicator utilized in the evaluation of the SANS.

In some embodiments, said evaluating the structural integrity of the ONS comprises obtaining changes in one or more of a surface area, a volume, a surface structure, and a pressure of the ONS by comparing the 3D images or the 3D derived quantitative measurements obtained with the provocative maneuver with the baseline.

In some embodiments, said determining if the living subject is at risk of developing the SANS comprises determining the development of the SANS, the efficacy of SANS treatments or countermeasures, and/or the ongoing injury during the spaceflight and/or on the space station in the low-or no-gravity environment based on the structural integrity of the ONS.

In some embodiments, the method further comprises obtaining 3D images or 3D quantitative measurements of the ONS of the living subject after returned to the Earth in a normal gravity environment; evaluating the 3D image or the 3D quantitative measurement obtained after returned on the Earth in the normal gravity environment by comparing them with the baseline; and assessing accumulated damage of the ONS in the low-or no-gravity environment from the baseline, based on the evaluation of the 3D images or the 3D quantitative measurements, and/or resolution of SANS predisposing factors after returned to the Earth in the normal gravity environment.

In some embodiments, the method for screening SANS comprises obtaining 3D images or 3D quantitative measurements of at least one ONS of the living subject at rest in a normal gravity environment prior to a spaceflight and during the spaceflight and/or on a space station in a low-or no-gravity environment, respectively, wherein the 3D images or the 3D quantitative measurements obtained at rest in the normal gravity environment is used as a baseline; evaluating the 3D image or the 3D quantitative measurement obtained during the spaceflight and/or on the space station in the low-or no-gravity environment by comparing them with the baseline; and determining a degree of predisposition to developing the SANS, an efficacy of SANS treatments or countermeasures, and/or an ongoing injury during the spaceflight and/or on the space station in the low-or no-gravity environment based on the evaluation of the 3D images or the 3D quantitative measurements.

In some embodiments, the 3D images are 3D ultrasound images obtained by an ultrasound probe, and wherein the 3D quantitative measurements are obtained by an MRI or CT scanner.

In some embodiments, said evaluating the 3D images or the 3D derived quantitative measurements comprises evaluating a structural integrity of the ONS using the 3D images or the 3D quantitative measurements obtained with the provocative maneuver, by comparing them with the baseline, wherein the structural integrity is a diagnostic and prognostic indicator utilized in the evaluation of the SANS.

In some embodiments, said evaluating the structural integrity of the ONS comprises obtaining changes in one or more of a surface area, a volume, a surface structure, and a pressure of the ONS by comparing the 3D images or the 3D derived quantitative measurements obtained with the provocative maneuver with the baseline.

In some embodiments, said obtaining the changes in the surface structure of the ONS comprises obtaining a degree of irregularity of the surface structure of the ONS visualized in the 3D images or the 3D derived quantitative measurements obtained with the provocative maneuver.

In some embodiments, the irregularity of the surface structure of the ONS includes bulges, wrinkles, and/or ripples formed on the exterior surface of the ONS.

In some embodiments, the degree of irregularity of the surface structure of the ONS is associated with a severity of a structural damage of the ONS, wherein the severity of the structural damage is proportional to the degree of the irregularity of the surface structure, and the more variability of the surface structure of the ONS, the more underlying structural damage and the higher the risk of developing the SANS in a low-or no-gravity environment.

In some embodiments, the variability of the surface structure of the ONS is measured by one or more of a mean radius at a specified point in the ONS distal to the retina and a standard deviation in the radii; a volume at a specific level measured posterior to the retina; and a volume of the ONS compared to an ideal cylinder.

In some embodiments, said determining if the living subject is at risk of developing the SANS comprises determining a degree of predisposition to developing the SANS based on the structural integrity of the ONS.

In some embodiments, the degree of predisposition to developing the SANS includes a relative propensity of developing the SANS based on exposure to a low-or zero-gravity environment.

In some embodiments, said determining the degree of predisposition to developing the SANS comprises correlating a dose of low gravity and/or a duration of exposing to a low-or zero-gravity environment to the risk of developing the SANS.

In some embodiments, the method further comprises obtaining 3D images or 3D quantitative measurements of the ONS of the living subject after returned to the Earth in a normal gravity environment; evaluating the 3D image or the 3D quantitative measurement obtained after returned on the Earth in the normal gravity environment by comparing them with the baseline; and assessing accumulated damage of the ONS in the low-or no-gravity environment from the baseline, based on the evaluation of the 3D images or the 3D quantitative measurements, and/or resolution of SANS predisposing factors after returned to the Earth in the normal gravity environment.

In some embodiments, the system for screening SANS comprises a means for obtaining 3D images or 3D quantitative measurements of at least one ONS of the living subject at rest and with a provocative maneuver, respectively, prior to a spaceflight, wherein the 3D images or the 3D quantitative measurements obtained at rest is used as a baseline; and a processor for evaluating the 3D images or the 3D quantitative measurements obtained at rest and with the provocative maneuver; and determining if the living subject is at risk of developing the SANS based on the evaluation of the 3D images or the 3D quantitative measurements.

In some embodiments, the provocative maneuver is an activity that increases the volume in the ONS, causing the ONS to dilate with minimal increases in the ICP.

In some embodiments, the provocative maneuver is a Valsalva maneuver.

In some embodiments, the means comprises an ultrasound probe, an MRI scanner, or a CT scanner.

In some embodiments, the ultrasound probe is a portable ultrasound using three linear arrays to simultaneously images of the ONS allowing for reconstruction of the 3D image.

In some embodiments, said evaluating the 3D images or the 3D derived quantitative measurements comprises evaluating a structural integrity of the ONS using the 3D images or the 3D quantitative measurements obtained with the provocative maneuver, by comparing them with the baseline, wherein the structural integrity is a diagnostic and prognostic indicator utilized in the evaluation of the SANS.

In some embodiments, said evaluating the structural integrity of the ONS comprises obtaining changes in one or more of a surface area, a volume, a surface structure, and a pressure of the ONS by comparing the 3D images or the 3D derived quantitative measurements obtained with the provocative maneuver with the baseline.

In some embodiments, said obtaining the changes in the surface structure of the ONS comprises obtaining a degree of irregularity of the surface structure of the ONS visualized in the 3D images or the 3D derived quantitative measurements obtained with the provocative maneuver.

In some embodiments, the irregularity of the surface structure of the ONS includes bulges, wrinkles, and/or ripples formed on the exterior surface of the ONS.

In some embodiments, the degree of irregularity of the surface structure of the ONS is associated with a severity of a structural damage of the ONS, wherein the severity of the structural damage is proportional to the degree of the irregularity of the surface structure, and the more variability of the surface structure of the ONS, the more underlying structural damage and the higher the risk of developing the SANS in a low-or no-gravity environment.

In some embodiments, the variability of the surface structure of the ONS is measured by one or more of a mean radius at a specified point in the ONS distal to the retina and a standard deviation in the radii; a volume at a specific level measured posterior to the retina; and a volume of the ONS compared to an ideal cylinder.

In some embodiments, said determining if the living subject is at risk of developing the SANS comprises determining a degree of predisposition to developing the SANS based on the structural integrity of the ONS.

In some embodiments, the degree of predisposition to developing the SANS includes a relative propensity of developing the SANS based on exposure to a low-or zero-gravity environment.

In some embodiments, said determining the degree of predisposition to developing the SANS comprises correlating a dose of low gravity and/or a duration of exposing to a low-or zero-gravity environment to the risk of developing the SANS.

In some embodiments, the means is further adapted for obtaining 3D images or 3D quantitative measurements of the ONS of the living subject during the spaceflight and/or on a space station in a low-or no-gravity environment; evaluating the 3D image or the 3D quantitative measurement obtained during the spaceflight and/or on the space station in the low-or no-gravity environment by comparing them with the baseline; and determining a development of the SANS, an efficacy of SANS treatments or countermeasures, and/or an ongoing injury during the spaceflight and/or on the space station in the low-or no-gravity environment based on the evaluation of the 3D images or the 3D quantitative measurements.

In some embodiments, said evaluating the 3D images or the 3D derived quantitative measurements comprises evaluating a structural integrity of the ONS using the 3D images or the 3D quantitative measurements obtained during the spaceflight and/or on the space station in the low-or no-gravity environment, by comparing them with the baseline, wherein the structural integrity is a diagnostic and prognostic indicator utilized in the evaluation of the SANS.

In some embodiments, said evaluating the structural integrity of the ONS comprises obtaining changes in one or more of a surface area, a volume, a surface structure, and a pressure of the ONS by comparing the 3D images or the 3D derived quantitative measurements obtained with the provocative maneuver with the baseline.

In some embodiments, said determining if the living subject is at risk of developing the SANS comprises determining the development of the SANS, the efficacy of SANS treatments or countermeasures, and/or the ongoing injury during the spaceflight and/or on the space station in the low-or no-gravity environment based on the structural integrity of the ONS.

In some embodiments, the means is further adapted for obtaining 3D images or 3D quantitative measurements of the ONS of the living subject after returned to the Earth in a normal gravity environment; evaluating the 3D image or the 3D quantitative measurement obtained after returned on the Earth in the normal gravity environment by comparing them with the baseline; and assessing accumulated damage of the ONS in the low-or no-gravity environment from the baseline, based on the evaluation of the 3D images or the 3D quantitative measurements, and/or resolution of SANS predisposing factors after returned to the Earth in the normal gravity environment.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. National Aeronautics and Space Administration, SANS Evidence Report, Nov. 30, 2017, https://humanresearchroadmap.nasa.gov/evidence/reports/SANS.pdf.
[2]. Lee, A.G. et al., Spaceflight associated neuro-ocular syndrome (SANS) and the neuro-ophthalmologic effects of microgravity: a review and an update. npj Microgravity 6, 7 (2020). https://doi.org/10.1038/s41526-020-0097-9.
[3]. East, L. Agrawal, P. Islam, Z. Hockman, T. Heger, I. Kuchinski, A.M. Gibson, R. Lyon, M. Ultrasound of the Optic Nerve Sheath as an Indicator of Mild Traumatic Brain Injury: Animal Model Provides Anatomical Basis. Annals of Emergency Medicine. 2018, 72:4, S47-S48.
[4]. Lyon, M. Agrawal P. Friez K. Gordon R. Morales I. Zhang L. Heger I. Gibson R. Effect of History of Mild Traumatic Brain Injury on Optic Nerve Sheath Diameter Changes after Valsalva Maneuver. Journal of Neurotrauma. 2018, 35:4, 695-702.
[5]. Super C. East L. Agrawal P. Dement M. Heger I. Gibson R. Lyon M. Increased Optic Nerve Sheath Diameter after Mild Traumatic Brain Injury in a High-Risk Population. Neurology. 2019, 92 (15 Supplement).

1. A method for screening spaceflight-associated neuro-ocular syndrome (SANS) for a living subject, comprising:
obtaining three-dimensional (3D) images or 3D quantitative measurements of at least one optic nerve sheath (ONS) of the living subject at rest and with a provocative maneuver, respectively, prior to a spaceflight, wherein the 3D images or the 3D quantitative measurements obtained at rest is used as a baseline;
evaluating the 3D images or the 3D quantitative measurements obtained at rest and with the provocative maneuver; and
determining if the living subject is at risk of developing the SANS based on the evaluation of the 3D images or the 3D quantitative measurements.

2. The method of claim 1, wherein the provocative maneuver is an activity that increases the volume in the ONS, causing the ONS to dilate with minimal increases in the intracranial pressure (ICP).

3. The method of claim 2, wherein the provocative maneuver is a Valsalva maneuver.

4. The method of claim 1, wherein the 3D images are 3D ultrasound images obtained by an ultrasound probe, and wherein the 3D quantitative measurements are obtained by a magnetic resonance imaging (MRI) or computed tomography (CT) scanner.

5. The method of claim 1, wherein said evaluating the 3D images or the 3D derived quantitative measurements comprises:
evaluating a structural integrity of the ONS using the 3D images or the 3D quantitative measurements obtained with the provocative maneuver, by comparing them with the baseline, wherein the structural integrity is a diagnostic and prognostic indicator utilized in the evaluation of the SANS.

6. The method of claim 5, wherein said evaluating the structural integrity of the ONS comprises:
obtaining changes in a surface area of the ONS by comparing the 3D images or the 3D derived quantitative measurements obtained with the provocative maneuver with the baseline.

7. The method of claim 5, wherein said evaluating the structural integrity of the ONS comprises:
obtaining changes in a volume of the ONS by comparing the 3D images or the 3D derived quantitative measurements obtained with the provocative maneuver with the baseline.

8. The method of claim 5, wherein said evaluating the structural integrity of the ONS comprises:
obtaining changes in a surface structure of the ONS by comparing the 3D images or the 3D derived quantitative measurements obtained with the provocative maneuver with the baseline.

9. The method of claim 8, wherein said obtaining the changes in the surface structure of the ONS comprises:
obtaining a degree of irregularity of the surface structure of the ONS visualized in the 3D images or the 3D derived quantitative measurements obtained with the provocative maneuver.

10. The method of claim 9, wherein the irregularity of the surface structure of the ONS includes bulges, wrinkles, and/or ripples formed on the exterior surface of the ONS.

11. The method of claim 10, wherein the degree of irregularity of the surface structure of the ONS is associated with a severity of a structural damage of the ONS, wherein the severity of the structural damage is proportional to the degree of the irregularity of the surface structure, and the more variability of the surface structure of the ONS, the more underlying structural damage and the higher the risk of developing the SANS in a low-or no-gravity environment.

12. The method of claim 11, wherein the variability of the surface structure of the ONS is measured by one or more of:
a mean radius at a specified point in the ONS distal to the retina and a standard deviation in the radii;
a volume at a specific level measured posterior to the retina; and
a volume of the ONS compared to an ideal cylinder.

13. The method of claim 1, wherein the 3D images or the 3D quantitative measurements are of real-time elastography of the ONS, wherein said evaluating the 3D images or the 3D quantitative measurements comprises:
obtaining changes in a pressure of fluid in the ONS using the 3D images or the 3D quantitative measurements.

14. The method of claim 5, wherein said determining if the living subject is at risk of developing the SANS comprises:
determining a degree of predisposition to developing the SANS based on the structural integrity of the ONS.

15. The method of claim 14, wherein the degree of predisposition to developing the SANS includes a relative propensity of developing the SANS based on exposure to a low-or zero-gravity environment.

16. The method of claim 15, wherein said determining the degree of predisposition to developing the SANS comprises correlating a dose of low gravity and/or a duration of exposing to a low-or zero-gravity environment to the risk of developing the SANS.

17. The method of claim 1, further comprising:
obtaining 3D images or 3D quantitative measurements of the ONS of the living subject during the spaceflight and/or on a space station in a low-or no-gravity environment;
evaluating the 3D image or the 3D quantitative measurement obtained during the spaceflight and/or on the space station in the low-or no-gravity environment by comparing them with the baseline; and
determining a development of the SANS, an efficacy of SANS treatments or countermeasures, and/or an ongoing injury during the spaceflight and/or on the space station in the low-or no-gravity environment based on the evaluation of the 3D images or the 3D quantitative measurements.

18. The method of claim 17, wherein said evaluating the 3D images or the 3D derived quantitative measurements comprises:
evaluating a structural integrity of the ONS using the 3D images or the 3D quantitative measurements obtained during the spaceflight and/or on the space station in the low-or no-gravity environment, by comparing them with the baseline, wherein the structural integrity is a diagnostic and prognostic indicator utilized in the evaluation of the SANS.

19. The method of claim 18 wherein said evaluating the structural integrity of the ONS comprises:
obtaining changes in one or more of a surface area, a volume, a surface structure, and a pressure of the ONS by comparing the 3D images or the 3D derived quantitative measurements obtained with the provocative maneuver with the baseline.

20. The method of claim 18, wherein said determining if the living subject is at risk of developing the SANS comprises:
determining the development of the SANS, the efficacy of SANS treatments or countermeasures, and/or the ongoing injury during the spaceflight and/or on the space station in the low-or no-gravity environment based on the structural integrity of the ONS.

21. The method of claim 17, further comprising:
obtaining 3D images or 3D quantitative measurements of the ONS of the living subject after returned to the Earth in a normal gravity environment;
evaluating the 3D image or the 3D quantitative measurement obtained after returned on the Earth in the normal gravity environment by comparing them with the baseline; and
assessing accumulated damage of the ONS in the low-or no-gravity environment from the baseline, based on the evaluation of the 3D images or the 3D quantitative measurements, and/or resolution of SANS predisposing factors after returned to the Earth in the normal gravity environment.

22. A method for screening spaceflight-associated neuro-ocular syndrome (SANS) for a living subject, comprising:
obtaining three-dimensional (3D) images or 3D quantitative measurements of at least one optic nerve sheath (ONS) of the living subject at rest in a normal gravity environment prior to a spaceflight and during the spaceflight and/or on a space station in a low-or no-gravity environment, respectively, wherein the 3D images or the 3D quantitative measurements obtained at rest in the normal gravity environment is used as a baseline;
evaluating the 3D image or the 3D quantitative measurement obtained during the spaceflight and/or on the space station in the low-or no-gravity environment by comparing them with the baseline; and
determining a degree of predisposition to developing the SANS, an efficacy of SANS treatments or countermeasures, and/or an ongoing injury during the spaceflight and/or on the space station in the low-or no-gravity environment based on the evaluation of the 3D images or the 3D quantitative measurements.

23. The method of claim 22, wherein the 3D images are 3D ultrasound images obtained by an ultrasound probe, and wherein the 3D quantitative measurements are obtained by magnetic a resonance imaging (MRI) or computed tomography (CT) scanner.

24. The method of claim 1, wherein said evaluating the 3D images or the 3D derived quantitative measurements comprises:
evaluating a structural integrity of the ONS using the 3D images or the 3D quantitative measurements obtained with the provocative maneuver, by comparing them with the baseline, wherein the structural integrity is a diagnostic and prognostic indicator utilized in the evaluation of the SANS.

25. The method of claim 24, wherein said evaluating the structural integrity of the ONS comprises:
obtaining changes in one or more of a surface area, a volume, a surface structure, and a pressure of the ONS by comparing the 3D images or the 3D derived quantitative measurements obtained with the provocative maneuver with the baseline.

26. The method of claim 25, wherein said obtaining the changes in the surface structure of the ONS comprises:
obtaining a degree of irregularity of the surface structure of the ONS visualized in the 3D images or the 3D derived quantitative measurements obtained with the provocative maneuver.

27. The method of claim 26, wherein the irregularity of the surface structure of the ONS includes bulges, wrinkles, and/or ripples formed on the exterior surface of the ONS.

28. The method of claim 27, wherein the degree of irregularity of the surface structure of the ONS is associated with a severity of a structural damage of the ONS, wherein the severity of the structural damage is proportional to the degree of the irregularity of the surface structure, and the more variability of the surface structure of the ONS, the more underlying structural damage and the higher the risk of developing the SANS in a low-or no-gravity environment.

29. The method of claim 28, wherein the variability of the surface structure of the ONS is measured by one or more of:
a mean radius at a specified point in the ONS distal to the retina and a standard deviation in the radii;
a volume at a specific level measured posterior to the retina; and
a volume of the ONS compared to an ideal cylinder.

30. The method of claim 24, wherein said determining if the living subject is at risk of developing the SANS comprises:
determining a degree of predisposition to developing the SANS based on the structural integrity of the ONS.

31. The method of claim 30, wherein the degree of predisposition to developing the SANS includes a relative propensity of developing the SANS based on exposure to a low-or zero-gravity environment.

32. The method of claim 31, wherein said determining the degree of predisposition to developing the SANS comprises correlating a dose of low gravity and/or a duration of exposing to a low-or zero-gravity environment to the risk of developing the SANS.

33. The method of claim 22, further comprising:
obtaining 3D images or 3D quantitative measurements of the ONS of the living subject after returned to the Earth in a normal gravity environment;
evaluating the 3D image or the 3D quantitative measurement obtained after returned on the Earth in the normal gravity environment by comparing them with the baseline; and
assessing accumulated damage of the ONS in the low-or no-gravity environment from the baseline, based on the evaluation of the 3D images or the 3D quantitative measurements, and/or resolution of SANS predisposing factors after returned to the Earth in the normal gravity environment.

34. A system for screening spaceflight-associated neuro-ocular syndrome (SANS) for a living subject, comprising:
a means for obtaining three-dimensional (3D) images or 3D quantitative measurements of at least one optic nerve sheath (ONS) of the living subject at rest and with a provocative maneuver, respectively, prior to a spaceflight, wherein the 3D images or the 3D quantitative measurements obtained at rest is used as a baseline; and
a processor for evaluating the 3D images or the 3D quantitative measurements obtained at rest and with the provocative maneuver; and determining if the living subject is at risk of developing the SANS based on the evaluation of the 3D images or the 3D quantitative measurements.

35. The system of claim 34, wherein the provocative maneuver is an activity that increases the volume in the ONS, causing the ONS to dilate with minimal increases in the intracranial pressure (ICP).

36. The system of claim 35, wherein the provocative maneuver is a Valsalva maneuver.

37. The system of claim 34, wherein the means comprises an ultrasound probe, a magnetic resonance imaging (MRI) scanner, or a computed tomography (CT) scanner.

38. The system of claim 37, wherein the ultrasound probe is a portable ultrasound using three linear arrays to simultaneously images of the ONS allowing for reconstruction of the 3D image.

39. The system of claim 34, wherein said evaluating the 3D images or the 3D derived quantitative measurements comprises:
evaluating a structural integrity of the ONS using the 3D images or the 3D quantitative measurements obtained with the provocative maneuver, by comparing them with the baseline, wherein the structural integrity is a diagnostic and prognostic indicator utilized in the evaluation of the SANS.

40. The system of claim 39, wherein said evaluating the structural integrity of the ONS comprises:
obtaining changes in one or more of a surface area, a volume, a surface structure, and a pressure of the ONS by comparing the 3D images or the 3D derived quantitative measurements obtained with the provocative maneuver with the baseline.

41. The system of claim 40, wherein said obtaining the changes in the surface structure of the ONS comprises:
obtaining a degree of irregularity of the surface structure of the ONS visualized in the 3D images or the 3D derived quantitative measurements obtained with the provocative maneuver.

42. The system of claim 41, wherein the irregularity of the surface structure of the ONS includes bulges, wrinkles, and/or ripples formed on the exterior surface of the ONS.

43. The system of claim 42, wherein the degree of irregularity of the surface structure of the ONS is associated with a severity of a structural damage of the ONS, wherein the severity of the structural damage is proportional to the degree of the irregularity of the surface structure, and the more variability of the surface structure of the ONS, the more underlying structural damage and the higher the risk of developing the SANS in a low-or no-gravity environment.

44. The system of claim 43, wherein the variability of the surface structure of the ONS is measured by one or more of:
a mean radius at a specified point in the ONS distal to the retina and a standard deviation in the radii;
a volume at a specific level measured posterior to the retina; and
a volume of the ONS compared to an ideal cylinder.

45. The system of claim 40, wherein said determining if the living subject is at risk of developing the SANS comprises:
determining a degree of predisposition to developing the SANS based on the structural integrity of the ONS.

46. The system of claim 45, wherein the degree of predisposition to developing the SANS includes a relative propensity of developing the SANS based on exposure to a low-or zero-gravity environment.

47. The system of claim 46, wherein said determining the degree of predisposition to developing the SANS comprises correlating a dose of low gravity and/or a duration of exposing to a low-or zero-gravity environment to the risk of developing the SANS.

48. The system of claim 34, wherein the means is further adapted for:
obtaining 3D images or 3D quantitative measurements of the ONS of the living subject during the spaceflight and/or on a space station in a low-or no-gravity environment;
evaluating the 3D image or the 3D quantitative measurement obtained during the spaceflight and/or on the space station in the low-or no-gravity environment by comparing them with the baseline; and
determining a development of the SANS, an efficacy of SANS treatments or countermeasures, and/or an ongoing injury during the spaceflight and/or on the space station in the low-or no-gravity environment based on the evaluation of the 3D images or the 3D quantitative measurements.

49. The system of claim 48, wherein said evaluating the 3D images or the 3D derived quantitative measurements comprises:
evaluating a structural integrity of the ONS using the 3D images or the 3D quantitative measurements obtained during the spaceflight and/or on the space station in the low-or no-gravity environment, by comparing them with the baseline, wherein the structural integrity is a diagnostic and prognostic indicator utilized in the evaluation of the SANS.

50. The system of claim 49 wherein said evaluating the structural integrity of the ONS comprises:
obtaining changes in one or more of a surface area, a volume, a surface structure, and a pressure of the ONS by comparing the 3D images or the 3D derived quantitative measurements obtained with the provocative maneuver with the baseline.

51. The system of claim 49, wherein said determining if the living subject is at risk of developing the SANS comprises:
determining the development of the SANS, the efficacy of SANS treatments or countermeasures, and/or the ongoing injury during the spaceflight and/or on the space station in the low-or no-gravity environment based on the structural integrity of the ONS.

52. The system of claim 48, further comprising:
obtaining 3D images or 3D quantitative measurements of the ONS of the living subject after returned to the Earth in a normal gravity environment;
evaluating the 3D image or the 3D quantitative measurement obtained after returned on the Earth in the normal gravity environment by comparing them with the baseline; and
assessing accumulated damage of the ONS in the low-or no-gravity environment from the baseline, based on the evaluation of the 3D images or the 3D quantitative measurements, and/or resolution of SANS predisposing factors after returned to the Earth in the normal gravity environment.
